(19) United States
(12) Patent Application Publication
(10) Pub. No.: US 2012/0309722 A1
(43) Pub. Date: Dec. 6, 2012

(54) THERAPEUTIC REGIMENS FOR THE TREATMENT OF IMMUNOINFLAMMATORY DISORDERS

(76) Inventor: Mahesh Padval, Needham, MA (US)

(21) Appl. No.: 13/587,503

(22) Filed: Aug. 16, 2012

Related U.S. Application Data
(63) Continuation of application No. 12/283,989, filed on Sep. 17, 2008.
(60) Provisional application No. 60/994,421, filed on Sep. 19, 2007.

(51) Int. Cl. A61K 31/573; A61K 31/56; A61P 37/02; A61K 31/58; A61P 29/00

(52) U.S. Cl. 514/170; 514/179; 514/174; 514/176; 514/171; 514/180

(57) ABSTRACT

The invention features pharmaceutical compositions and dosing regimens for the treatment of immunoinflammatory disorders.

### CROSS-REFERENCE TO RELATED APPLICATIONS

This application is a continuation of U.S. patent application Ser. No. 12/283,989, filed on Sep. 17, 2008, which claims the benefit from U.S. Ser. No. 60/994,421, filed Sep. 19, 2007. The disclosure of each application is incorporated by reference in their entirety.

### BACKGROUND OF THE INVENTION

The combination of prednisolone and dipyridamole is an orally available synergistic drug candidate in Phase 2 clinical development for the treatment of immunoinflammatory disorders. A synergistic drug includes two compounds that are designed to act synergistically through multiple pathways to provide a therapeutic effect which neither component administered alone and at the same dosing levels can achieve. The combination of prednisolone with dipyridamole was designed to selectively amplify certain elements of prednisolone's anti-inflammatory and immunomodulatory activities, without replicating steroid side effects.

Proper formulation is essential to maximize the therapeutic benefit of a synergistic drug combination.

### SUMMARY OF THE INVENTION

The invention provides methods, compositions, and kits for administering dipyridamole in combination with a corticosteroid. This combination is useful for the treatment of immunoinflammatory disorders.

Accordingly, in a first aspect, the invention features a method for treating an immunoinflammatory disorder in a subject in need thereof by (i) administering to the subject a first dose of corticosteroid at time T₀; and (ii) administering to the subject a second dose of corticosteroid 3 to 8 hours after time T₀, wherein the ratio of the first dose to the second dose is 1.5-2.5:1. In certain embodiments, the ratio of the first dose to the second dose is 1.5:1, 1.6:1, 1.7:1, 1.8:1, 1.9:1, 2.0:1, 2.1:1, 2.2:1, 2.3:1, 2.4:1, or even 2.5:1. In other embodiments, the first dose is administered in a unit dosage formulation including from 1 to 10 mg, desirably 1 to 8 mg, 1 to 5 mg, 1.25 to 3 mg, 1.4 to 2.3 mg, or 1.5 to 2.5 mg of prednisolone or an equivalent, equipotent amount of another corticosteroid, and the second dose is administered in a unit dosage formulation including from 0.5 to 5 mg, desirably from 0.5 to 4 mg, 0.5 to 3 mg, 0.5 to 2 mg, 0.75 to 2 mg, 0.70 to 1.20 mg, or 0.75 to 1.25 mg of prednisolone or an equivalent, equipotent amount of another corticosteroid. In still other embodiments, the first dose is administered in a unit dosage formulation including 1.5, 1.6, 1.7, 1.8, 1.9, or 2.0 mg of prednisolone or an equivalent, equipotent amount of another corticosteroid, and the second dose is administered in a unit dosage formulation including 0.7, 0.8, 0.9, or 1.0 mg of prednisolone or an equivalent, equipotent amount of another corticosteroid. In certain embodiments, the corticosteroid is formulated for immediate release. The method can further include administering to the subject dipyridamole in unit dosage form (e.g., 40 to 200 mg, 40 to 180 mg, 45 to 200 mg, 50 to 200 mg, 70 to 200 mg, 90 to 200 mg, 90 to 180 mg, or 120 to 180 mg) of dipyridamole. In certain embodiments 180 mg, 120 mg, 90 mg, 60 mg, or 45 mg of dipyridamole in unit dosage form is administered to the subject.

In a related aspect the invention features a pharmaceutical composition in unit dosage form including (i) 1 to 10 mg, desirably 1 to 8 mg, 1 to 5 mg, 1.25 to 3 mg, 1.4 to 2.3 mg, or 1.5 to 2.5 mg of prednisolone or an equivalent, equipotent amount of another corticosteroid; and (ii) 40 to 200 mg, 40 to 180 mg, 45 to 200 mg, 50 to 200 mg, 70 to 200 mg, 90 to 200 mg, 90 to 180 mg, or 120 to 180 mg of dipyridamole. In certain embodiments, the pharmaceutical composition includes 1.5, 1.6, 1.7, 1.8, 1.9, or 2.0 mg of prednisolone or an equivalent, equipotent amount of another corticosteroid; and (ii) 180 mg, 120 mg, 90 mg, 60 mg, or 45 mg of dipyridamole. For example, the pharmaceutical composition can include 1.8 mg of prednisolone or an equivalent, equipotent amount of another corticosteroid; and (ii) 180 mg, 90 mg, or 45 mg of dipyridamole.

In invention further features a pharmaceutical composition in unit dosage form including (i) 0.5 to 5 mg, desirably from 0.5 to 4 mg, 0.5 to 3 mg, 0.5 to 2 mg, 0.75 to 2 mg, 0.70 to 1.20 mg, or 0.75 to 1.25 mg of prednisolone or an equivalent, equipotent amount of another corticosteroid; and (ii) 40 to 200 mg, 40 to 180 mg, 45 to 200 mg, 50 to 200 mg, 70 to 200 mg, 90 to 200 mg, 90 to 180 mg, or 120 to 180 mg of dipyridamole. In certain embodiments, the pharmaceutical composition includes 0.7, 0.8, 0.9, or 1.0 mg of prednisolone or an equivalent, equipotent amount of another corticosteroid; and (ii) 180 mg, 120 mg, 90 mg, 60 mg, or 45 mg of dipyridamole. For example, the pharmaceutical composition can include 0.9 mg of prednisolone or an equivalent, equipotent amount of another corticosteroid; and (ii) 180 mg, 90 mg, or 45 mg of dipyridamole.

In one embodiment, of the above aspects, each of the corticosteroid and the dipyridamole is formulated for immediate release. In another embodiment of the above aspects, the dipyridamole is formulated as a homogenous bead. In still another embodiment of the above aspects, the corticosteroid is formulated as a coated non-pareil bead.

In invention features a pharmaceutical composition in unit dosage form including homogenous dipyridamole beads. In certain embodiments, the unit dosage form includes from 40 to 200 mg, 40 to 180 mg, 45 to 200 mg, 50 to 200 mg, 70 to 200 mg, 90 to 200 mg, 90 to 180 mg, or 120 to 180 mg of dipyridamole. In other embodiments, the unit dosage form comprises 180 mg, 120 mg, 90 mg, 60 mg, or 45 mg of dipyridamole.

In another aspect, the invention features a kit including (i) a first pharmaceutical composition of the invention including prednisolone or an equivalent, equipotent amount of another corticosteroid, and dipyridamole; (ii) a second pharmaceutical composition of the invention including prednisolone or an equivalent, equipotent amount of another corticosteroid, and dipyridamole; and (iii) instructions for administering the second pharmaceutical composition 3 to 8 hours after the first pharmaceutical composition. In certain embodiments, the kit includes instructions for administering the second pharmaceutical composition 3 to 8, 3 to 7, 3 to 6, 4 to 8, 4 to 7, or 4 to 6 hours after the first pharmaceutical composition. In other embodiments, the kit includes instructions for administering the first pharmaceutical composition upon waking. In still other embodiments, the kit includes instructions for administering the first pharmaceutical composition and the second pharmaceutical composition for the treatment of an immunoinflammtory disease.

In a related aspect, the invention features a kit including (i) a first pharmaceutical composition in a unit dosage formulation including from 1 to 10 mg, desirably 1 to 8 mg, 1 to 5 mg, 1.25 to 3 mg, 1.4 to 2.3 mg, or 1.5 to 2.5 mg of prednisolone or an equivalent, equipotent amount of another corticosteroid; and (ii) a second pharmaceutical composition in a unit dosage formulation comprising from 0.5 to 4 mg, 0.5 to 3 mg, 0.5 to 2 mg, 0.75 to 2 mg, 0.70 to 1.20 mg, or 0.75 to 1.25 mg of prednisolone or an equivalent, equipotent amount of another corticosteroid; and (iii) instructions for administering the second pharmaceutical composition 3 to 8 hours after the first pharmaceutical composition. In certain embodiments, the first pharmaceutical composition includes 1.5, 1.6, 1.7, 1.8, 1.9, or 2.0 mg of prednisolone or an equivalent, equipotent amount of another corticosteroid, and the second pharmaceutical composition includes 0.7, 0.8, 0.9, or 1.0 mg of prednisolone or an equivalent, equipotent amount of another corticosteroid. In certain embodiments, the corticosteroid is formulated for immediate release. Each of the first pharmaceutical composition and the second pharmaceutical composition can further include dipyridamole (e.g., 40 to 200 mg, 40 to 180 mg, 45 to 200 mg, 50 to 200 mg, 70 to 200 mg, 90 to 200 mg, 90 to 180 mg, or 120 to 180 mg). In certain embodiments each of the first pharmaceutical composition and the second pharmaceutical composition include 180 mg, 120 mg, 90 mg, 60 mg, or 45 mg of dipyridamole.

In certain embodiments, the kits of the invention include instructions for administering the first pharmaceutical composition and the second pharmaceutical composition for the treatment of an immunoinflammtory disease.

In any of the above methods, compositions, and kits the corticosteroid can be, without limitation, selected from prednisolone, prednisone, budesonide, methylprednisolone, fluticasone, betamethasone, and deflazacort.

In any of the above methods and kits the first dose of corticosteroid can be, for example, administered to the subject upon waking (e.g., time T₀), while the second dose is administered to the subject, for example, 3 to 8, 3 to 7, 3 to 6, 4 to 8, 4 to 7, or 4 to 6 hours after time T₀.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-1 diabetes; ulcerative colitis; uveitis; vasculitis; and Wegener's granulomatosis.

By “corticosteroid” is meant any naturally occurring or synthetic steroid hormone which can be derived from cholesterol and is characterized by a hydrogenated cyclopentanoperhydrophenanthrene ring system. Naturally occurring corticosteroids are generally produced by the adrenal cortex. Synthetic corticosteroids may be halogenated. Functional groups required for activity include a double bond at Δ4, a C3 ketone, and a C20 ketone. Corticosteroids may have glucocorticoid and/or mineralocorticoid activity. In preferred embodiments, the corticosteroid is either fludrocortisone or prednisolone. Exemplary corticosteroids are 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin. Desirably, the corticosteroid is fludrocortisone or prednisolone.

By “an effective amount” is meant the amount of a compound, in a combination of the invention, required to treat or prevent an immunoinflammatory disorder. The effective amount of active compound(s) used to practice the present invention for therapeutic treatment of conditions caused by or contributing to an inflammatory disease varies depending upon the manner of administration, the age, body weight, and general health of the patient. Ultimately, the attending physician or veterinarian will decide the appropriate amount and dosage regimen. Such amount is referred to as an effective amount.

By an “equivalent, equipotent amount” is meant a dosage of a corticosteroid that produces the same anti-inflammatory effect in a patient as a recited dosage of prednisolone.

By “immediate release” is meant that the therapeutically active component (e.g., a corticosteroid) is released from the formulation immediately such that 80%, 85%, 90%, or even 95% of the component in the formulation is absorbed into the blood stream of a patient less than two hours after administration. Whether a pharmaceutical composition is formulated for immediate release can be determined by measuring the pharmacokinetic profile of the formulation.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, ascorbate, aspartate, benzoate, citrate, digluconate, ftimarate, glucoheptonate, glycerophosphate, hemisulfate, heptonate, hexanoate, hydrobromide, hydrochloride, hydroiodide, lactate, malate, maleate, malonate, mesylate, oxalate, phosphate, succinate, sulfate, tartrate, thiocyanate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “unit dosage form” and “unit dosage formulation” refer to physically discrete units suitable as unitary dosages, such as a pill, tablet, caplet, hard capsule, or soft capsule, each unit containing a predetermined quantity of dipyridamole and/or corticosteroid.

As used herein, the term “homogeneous bead” refers to a bead formulation including dipyridamole distributed throughout the bead along with other pharmaceutically acceptable excipients (e.g., diluents and binders). Homogeneous beads can be prepared as described in the examples.

As used herein, the term “coated” refers to a bead formulation including a corticosteroid, such as prednisolone, applied to the surface of a carrier, such as a non-pareil seed. Coated beads can be prepared as described in the examples.

Other features and advantages of the invention will be apparent from the following detailed description, the drawings, and the claims.

### BRIEF DESCRIPTION OF THE DRAWINGS

FIG. 1 is a flow chart depicting the prednisolone bead manufacturing process.

FIG. 2 is a flow chart depicting the dipyridamole bead manufacturing process.

FIG. 3 is a flow chart depicting the dipyridamole/prednisolone capsule manufacturing process.

### DETAILED DESCRIPTION

The invention provides for pharmaceutical compositions in unit dosage form containing dipyridamole and a corticosteroid. The compositions are useful for the treatment of immunoinflammatory disorders.

### Corticosteroids

The combinations of the invention include a corticosteroid selected from the class of selective glucocorticosteroid receptor agonists (SEGRAs) including, without limitation, 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdys one; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Standard recommended dosages for various steroid/disease combinations are provided in Table 1, below.

TABLE 1
Standard Recommended Corticosteroid Dosages

| Indication | Route | Drug | Dose | | Schedule |
|---|---|---|---|---|---|
| Psoriasis | oral | Prednisolone | 7.5-60 | mg | per day or divided b.i.d. |
| | oral | Prednisone | 7.5-60 | mg | per day or divided b.i.d. |
| Asthma | inhaled | beclomethasone dipropionate | 42 | μg/puff) | 4-8 puffs b.i.d. |
| | inhaled | Budesonide | (200 | μg/inhalation) | 1-2 inhalations b.i.d. |
| | inhaled | Flunisolide | (250 | μg/puff) | 2-4 puffs b.i.d. |
| | inhaled | fluticasone propionate | (44, 110 or 220 | μg/puff) | 2-4 puffs b.i.d. |
| | inhaled | triamcinolone acetonide | (100 | μg/puff) | 2-4 puffs b.i.d. |
| COPD | oral | Prednisone | 30-40 | mg | per day |
| Crohn's disease | oral | Budesonide | 9 | mg | per day |
| Ulcerative colitis | oral | Prednisone | 40-60 | mg | per day |
| | oral | Hydrocortisone | 300 | mg (IV) | per day |
| | oral | Methylprednisolone | 40-60 | mg | per day |
| Rheumatoid arthritis | oral | Prednisone | 10 | mg | per day |

Other standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone. Two or more corticosteroids can be administered in the same treatment.

Equivalent potency in clinical dosing is well known. Information relating to equivalent corticosteroid dosing may be found in the British National Formulary (BNF), 37 March 1999, the content of which is incorporated herein by reference.

The BNF guidelines are included in Table 2 below. More specifically, Table 2 provides doses of corticosteroids equivalent to 5 mg of prednisolone and equivalent to 1 mg of prednisolone when administered in a manner according to this invention.

TABLE 2
Equivalent Dose to Prednisolone

| | Equal to 5 mg | | Equal to 1 mg | |
|---|---|---|---|---|
| Drug | prednisolone | | prednisolone | |
| betamethasone | 750 | μg | 150 | μg |
| cortisone acetate | 25 | mg | 5 | mg |
| deflazacort | 6 | mg | 1.2 | mg |
| dexamethasone | 750 | μg | 150 | μg |
| hydrocortisone | 20 | mg | 4 | mg |
| methyl prednisone | 4 | mg | 0.8 | mg |
| triamcinolone | 4 | mg | 0.8 | mg |

It is also known (BNF 37 March 1999) from clinical dosing equivalence that doses of triamcinolone, fluticasone, and budesonide are broadly similar in nasal administration (110 μg, 100 μg, and 200 μg).

Two or more corticosteroids can be administered in the same treatment, or present in the same kit or unit dosage formulation.

### Formulation

The combination of the invention may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Formulations for oral use include tablets containing the active ingredient(s) in a mixture with non-toxic pharmaceutically acceptable excipients, preferably an excipient from the GRAS listing. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided in unit dosage form as chewable tablets, tablets, caplets, or capsules (e.g., as hard gelatin capsules wherein the active ingredient is mixed with an inert solid diluent, or as soft gelatin capsules wherein the active ingredient is mixed with water or an oil medium).

The formulations of the invention include diluents (e.g., lactose monohydrate, cellulose, glyceryl monostearate, and/or dibasic calcium phosphate, among others) and binders (e.g., polyvinylpyrrolidone, hypromellose, sucrose, guar gum, and/or starch). Any diluent or binder known in the art can be used in the methods, compositions, and kits of the invention.

### Kits

The individually or separately formulated agents of the invention can be packaged together, or individually, as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like. Kits may also include instructions for administering the pharmaceutical compositions using any indication and/or dosing regime described herein. Further description of kits is provided in the examples.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Drug Product

Dipyridamole and prednisolone were formulated in bead form and encapsulated in a standard size ‘0’ capsule. Six distinct capsule strengths were manufactured to accommodate the unequal amounts of prednisolone given in the morning and afternoon, and to allow for dose ranging. The doses of prednisolone and dipyridamole in the highest strength of each component (1.8 mg prednisolone+180 mg dipyridamole) were selected on the basis of the maximal quantities that could be filled into a size 0 capsule and have been shown to be efficacious in subjects with rheumatoid arthritis (RA) and osteoarthritis (OA), i.e., 2 mg prednisolone+200 mg dipyridamole at 0800 hours and 1 mg prednisolone+200 mg dipyridamole at 1300 hours. The strengths are shown in Table 3.

TABLE 3
Prednisolone and Dipyridamole Quantities in Capsules

| | Prednisolone | Dipyridamole |
|---|---|---|
| Dosing Time | Quantity/Capsule | Quantity/Capsule |
| 0800 hours | 1.8 mg | 45 mg |
| | 1.8 mg | 90 mg |
| | 1.8 mg | 180 mg |
| 1300 hours | 0.9 mg | 45 mg |
| | 0.9 mg | 90 mg |
| | 0.9 mg | 180 mg |

The quantitative composition of the capsules is provided in Table 4 and Table 5, where the first table gives the quantitative compositions of the three dosage strengths that contain 0.9 mg prednisolone with varying amounts of dipyridamole and the second table gives the quantitative compositions of the three dosage strengths that contain 1.8 mg prednisolone.

TABLE 4
Composition of Drug Product Dosage Form Containing 0.9 mg Prednisolone
Quantity per Capsule

| Ingredient | Function | Standard | 0.9/45 mg | 0.9/90 mg | 0.9/180 mg |
|---|---|---|---|---|---|
| Prednisolone anhydrous | Active | USP/EP | 0.90 mg | 0.90 mg | 0.90 mg |
| micronized | | | | | |
| Dipyridamole | Active | USP/EP/BP | 45.00 mg | 90.00 mg | 180.00 mg |
| Microcrystalline | Carrier for | USP/NF/EP | 87.03 mg | 87.03 mg | 87.03 mg |
| cellulose (Celphere CP- | prednisolone | | | | |
| 708) | | | | | |
| Microcrystalline | Diluent | USP/NF/EP | 11.30 mg | 22.54 mg | 45.10 mg |
| cellulose | | | | | |
| (Avicel PH 102) | | | | | |
| Polyvinylpyrrolidone | Binder | USP/EP | 3.29 mg | 5.99 mg | 11.39 mg |
| (Kollidon 30) | | | | | |
| Pregelatinized starch | Diluent, | USP | 11.30 mg | 22.54 mg | 45.10 mg |
| | binder | | | | |
| Purified water^{b} | Granulating | USP | QS | QS | QS |
| | agent | | | | |

^{b}Removed during processing
Abbreviations:
EP = European Pharmacopeia;
NF = National Formulary;
QS = quantity sufficient;
USP = United States Pharmacopeia

TABLE 5
Composition of Drug Product Dosage Form Containing 1.8 mg Prednisolone
Quantity per Capsule

| Ingredient | Function | Standard | 1.8/45 mg | 1.8/90 mg | 1.8/180 mg |
|---|---|---|---|---|---|
| Prednisolone anhydrous | Active | USP/EP | 1.80 mg | 1.80 mg | 1.80 mg |
| micronized | | | | | |
| Dipyridamole | Active | USP/EP/BP | 45.00 mg | 90.00 mg | 180.00 mg |
| Microcrystalline | Carrier for | USP/NF/EP | 87.03 mg | 87.03 mg | 87.03 mg |
| cellulose (Celphere CP- | prednisolone | | | | |
| 708) | | | | | |
| Microcrystalline | Diluent | USP/NF/EP | 11.30 mg | 22.54 mg | 45.10 mg |
| cellulose | | | | | |
| (Avicel PH 102) | | | | | |
| Polyvinylpyrrolidone | Binder | USP/EP | 3.87 mg | 6.57 mg | 11.97 mg |
| (Kollidon 30) | | | | | |
| Pregelatinized starch | Diluent, | USP | 11.30 mg | 22.54 mg | 45.10 mg |
| | binder | | | | |
| Purified water^{b} | Granulating | USP | QS | QS | QS |
| | agent | | | | |

^{b}Removed during processing
Abbreviations:
EP = European Pharmacopeia;
NF = National Formulary;
QS = quantity sufficient;
USP = United States Pharmacopeia

### Manufacturing Process

The manufacturing process for formulations of the combinations of the invention includes three manufacturing steps followed by packaging: the manufacture of prednisolone beads, the manufacture of dipyridamole beads, and the manufacture of capsules and packaging.

Prednisolone Bead Manufacturing Process

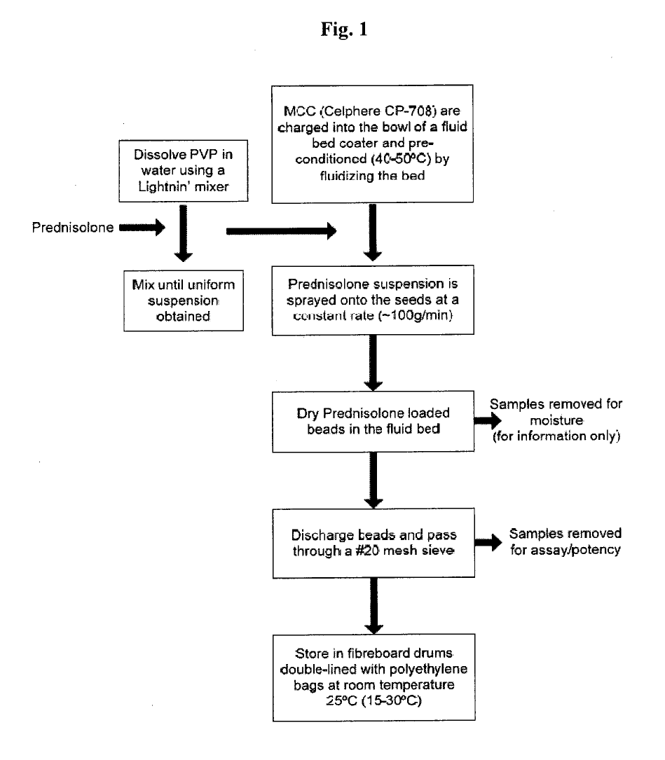

The prednisolone beads are manufactured by coating non-pareil seeds with prednisolone. The process is described in greater detail below and is shown schematically in FIG. 1. PVP (Kollidon 30) is dissolved in purified water using a Lightnin' mixer. Prednisolone is then added to the solution of PVP and water and mixed until a uniform suspension is formed. Non-pareil seeds of MCC (Celphere CP-708) are charged into the bowl of a fluid bed coater and pre-conditioned to temperature of 40-50° C. by fluidizing the bed. The prednisolone suspension is sprayed onto the fluidizing pre-conditioned non-pareil seeds at a constant rate of ˜100 g/minute ensuring that there is no agglomeration of the beads due to excessive wetting. Care is taken to ensure that an appropriate spray rate is maintained so as to prevent spray drying of prednisolone. The product bed temperature is maintained within the range of 40-50° C. by maintaining the inlet air temperature range of 60-70° C. Upon completion of the spray process, the prednisolone loaded beads are dried to a moisture content of less than 2%. The dried beads are discharged and screened through a #20 mesh sieve to remove any agglomerates. The screened beads are stored at room temperature 25° C. (15 to 30° C.) in fiber-board drums double lined with polyethylene bags. The prednisolone beads are analyzed for potency (assay) to determine the appropriate fill weight for the manufacture of the capsules. Table 6 summarizes the quantitative compositions of prednisolone capsules.

TABLE 6
Composition of Prednisolone Capsules

| Ingredient | Function | Standard | 0.9 mg | 1.8 mg |
|---|---|---|---|---|
| Prednisolone | Active | USP/EP | 0.9 mg | 1.80 mg |
| anhydrous | | | | |
| micronized | | | | |
| Microcrystalline | Carrier for | USP/NF/EP | 87.03 mg | 87.03 mg |
| cellulose | prednisolone | | | |
| (Celphere CP-708) | | | | |
| Polyvinyl- | Binder | USP/EP | 0.585 mg | 1.17 mg |
| pyrrolidone | | | | |
| (Kollidon 30) | | | | |
| Purified water^{b} | Granulating | USP | QS | QS |
| | agent | | | |

^{b}Removed during processing
Abbreviations:
EP = European Pharmacopeia;
NF = National Formulary;
QS = quantity sufficient;
USP = United States Pharmacopeia

Dipyridamole Bead Manufacturing Process

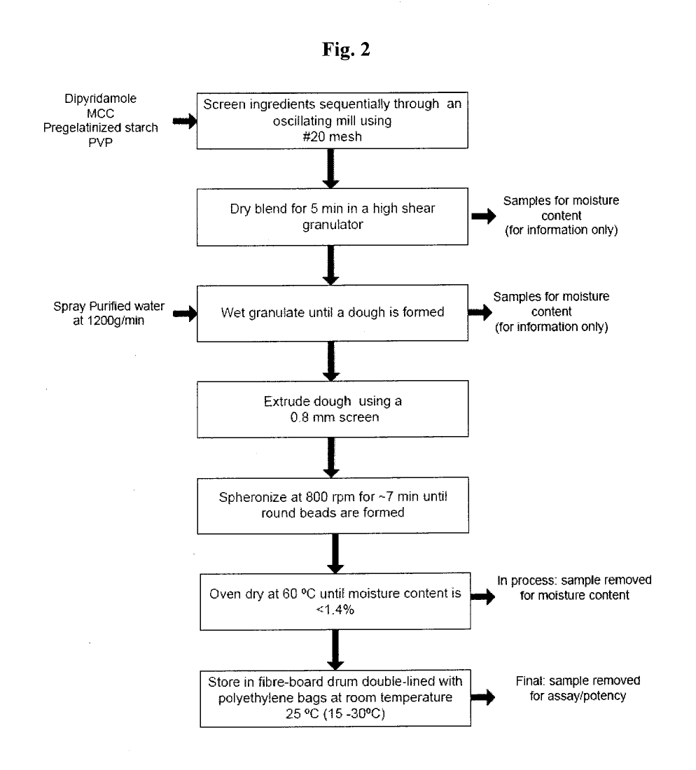

The dipyridamole beads are manufactured by extrusion spheronization. The manufacturing process for the dipyridamole beads is described in greater detail below and is shown schematically in FIG. 2. Dipyridamole is screened using an oscillating mill fitted with a #20 mesh screen and transferred into the bowl of a high shear granulator. MCC, pregelatinized starch and PVP are added to the oscillating mill successively to wash out any remaining dipyridamole. The milled materials are transferred into the bowl of a high shear granulator where they are dry blended for 5 minutes. A moisture sample of the dry blend is taken for information purposes only. The dry dipyridamole mix is then wet granulated using purified water as the granulating agent at a spray rate of 1200 g/minute till a dough is formed. Samples are removed for determination of moisture content. The wet mass of the dipyridamole dough is passed through the 0.8 mm screen of the extruder and spheronized for about 7 minutes at 800 revolutions per minute (rpm) until rounded beads are formed. The wet beads are dried in an oven set at 60° C. until the moisture content is less than 1.4%. The dried beads are stored at room temperature 25° C. (15-30° C.) in fiber-board drums double lined with polyethylene bags. The final beads are analyzed for potency (assay) to determine the appropriate fill weight for capsules. Table 7 summarizes the quantitative compositions of dipyridamole capsules.

TABLE 7
Composition of Dipyridamole Capsules
Quantity per Capsule

| Ingredient | Function | Standard | 45 mg | 90 mg | 180 mg |
|---|---|---|---|---|---|
| Dipyridamole | Active | USP/EP/BP | 45.00 mg | 90.00 mg | 180.00 mg |
| Microcrystalline cellulose | Diluent | USP/NF/EP | 11.30 mg | 22.54 mg | 45.10 mg |
| (Avicel PH 102) | | | | | |
| Pregelatinized starch | Diluent, | USP | 11.30 mg | 22.54 mg | 45.10 mg |
| | binder | | | | |
| Polyvinylpyrrolidone | Binder | USP/EP | 2.70 mg | 5.40 mg | 10.80 mg |
| (Kollidon 30) | | | | | |
| Microcrystalline cellulose | filler | USP/NF/EP | 100 mg | — | — |
| (Celphere CP-708) | | | | | |
| Purified water^{b} | Granulating | USP | QS | QS | QS |
| | agent | | | | |

^{b}Removed during processing
Abbreviations:
EP = European Pharmacopeia;
NF = National Formulary;
QS = quantity sufficient;
USP = United States Pharmacopeia

Capsule Manufacturing Process

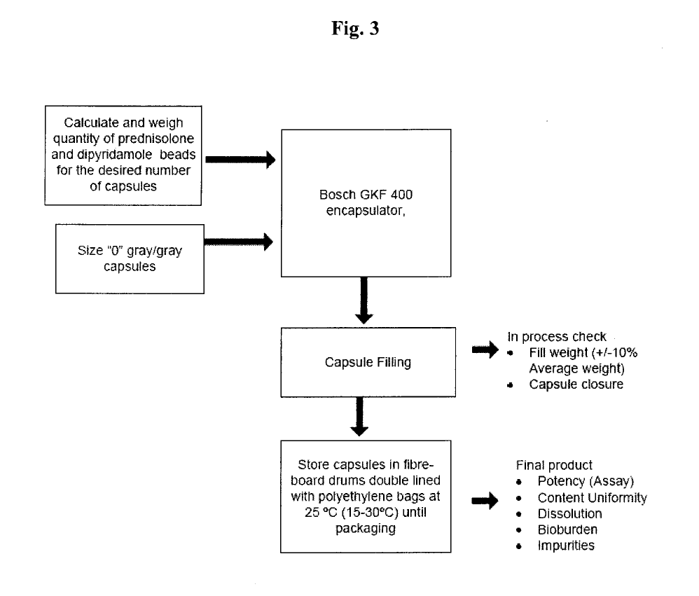

The capsule manufacturing process is described below and shown schematically in FIG. 3. The fill weight of each capsule is calculated based upon the percent weight/weight potency values of the prednisolone and dipyridamole beads. The quantity of each type of bead for the desired number of capsules is weighed and added to the Bosch GKF 400 encapsulator along with empty capsules. The prednisolone and dipyridamole beads are filled into size “0” gray/gray capsules. During the encapsulation process, capsules are checked at pre-determined intervals for fill weight variation and proper capsule closure. The machine is adjusted if any deviation is found in the established fill weight. The filled capsules are stored at room temperature conditions of 25° C. (15 to 30° C.) in fiber-board drums double lined with polyethylene bags. The final capsules are tested for identity of the active ingredients, potency of prednisolone and dipyridamole, content uniformity, dissolution, presence and quantities of related substances and bioburden prior to release.

Packaging

Dipyridamole/prednisolone capsules are packaged in blister packs using an Uhlman packaging machine. Bulk capsules are placed on a tray of the Uhlman packager to flood feed the blister cavities. The sealing layers are placed over strips containing five capsules each and are heat sealed into place. The sealed strips are inspected at the beginning and end of the process and at 30 minute intervals during the process for proper seals and missed cavities and placed into a labeled holding container if found satisfactory. The holding container is stored in the warehouse for secondary packaging.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for treating an immunoinflammatory disorder in a subject in need thereof, said method comprising:
i) administering to said subject a first dose of corticosteroid at time T₀; and
ii) administering to said subject a second dose of corticosteroid 3 to 8 hours after time T₀,
wherein the ratio of said first dose to said second dose is 1.5-2.5:1.

2. The method of claim 1, wherein the ratio of said first dose to said second dose is 2:1.

3. The method of claim 1, wherein said first dose is administered in a unit dosage formulation comprising from 1.4 to 2.3 mg of prednisolone or an equivalent, equipotent amount of another corticosteroid, and said second dose is administered in a unit dosage formulation comprising from 0.70 to 1.20 mg of prednisolone or an equivalent, equipotent amount of another corticosteroid.

4. The method of claim 3, wherein said first dose is administered in a unit dosage formulation comprising 1.8 mg of prednisolone or an equivalent, equipotent amount of another corticosteroid, and said second dose is administered in a unit dosage formulation comprising 0.9 mg of prednisolone or an equivalent, equipotent amount of another corticosteroid.

5. The method of claim 1, wherein said corticosteroid is selected from prednisolone, prednisone, budesonide, methylprednisolone, fluticasone, betamethasone, and deflazacort.

6. The method of claim 1, wherein said first dose is administered to said subject upon waking.

7. The method of claim 1, wherein said second dose is administered to said subject 4 to 6 hours after time T₀.

8. The method of claim 1, wherein said corticosteroid is formulated for immediate release.

9. The method of claim 1, further comprising administering to said subject dipyridamole in unit dosage form.

10. The method of claim 9, wherein said unit dosage form comprises from 40 to 200 mg of dipyridamole.

11. The method of claim 9, wherein said dipyridamole is formulated for immediate release.

12. A pharmaceutical composition in unit dosage form comprising (i) 1.4 to 2.3 mg of prednisolone or an equivalent, equipotent amount of another corticosteroid; and (ii) 40 to 200 mg of dipyridamole.

13. The pharmaceutical composition of claim 12, wherein each of said corticosteroid and said dipyridamole is formulated for immediate release.

14. The pharmaceutical composition of claim 12, wherein said dipyridamole is formulated as a homogenous bead.

15. A pharmaceutical composition in unit dosage form comprising (i) 0.70 to 1.20 mg of prednisolone or an equivalent, equipotent amount of another corticosteroid; and (ii) 40 to 200 mg of dipyridamole.

16. The pharmaceutical composition of claim 15, wherein each of said corticosteroid and said dipyridamole is formulated for immediate release.

17. The pharmaceutical composition of claim 15, wherein said dipyridamole is formulated as a homogenous bead.
